(19) United States
(12) Patent Application Publication
(10) Pub. No.: US 2026/0177536 A1
(43) Pub. Date: Jun. 25, 2026

(54) METHOD AND COMPUTER PROGRAM FOR CALIBRATING AN ELECTRONIC DEVICE FOR CHARACTERIZING A FLUID, AND CORRESPONDING ELECTRONIC DEVICE

(71) Applicant: ARYBALLE, Grenoble (FR)

(72) Inventors: Van Tri NGUYEN, Grenoble (FR); Yanis CARITU, Grenoble (FR); Pierre MAHO, Grenoble (FR)

(21) Appl. No.: 18/873,093

(22) PCT Filed: Jun. 1, 2023
(86) PCT No.: PCT/IB2023/055646

§ 371 Date: Mar. 3, 2026

(30) Foreign Application Priority Data
Jun. 10, 2022 (FR) FR2205630

(51) Int. Cl. G01N 33/00; G01N 21/552

(52) U.S. Cl. CPC G01N 33/0008; G01N 21/553; G01N 2201/12753

(57) ABSTRACT

This method for calibrating an electronic device for characterizing a fluid comprises storing multiple reference calibration signatures (YREF_{r,i}) and a parametric drift model (MOD(α,t)). For each of multiple calibration fluids, a calibration signature (YDEV_{r,i}) is obtained through interaction with the device. At least one parameter value (αᵣ,tᵣ) is determined by comparing the obtained calibration signatures (YDEV_{r,i}) with the reference calibration signatures (YREF_{r,i}). The calibration fluids are more precisely distributed into multiple calibration groups and parameter values are determined specifically for each calibration group.

The present invention relates to a method of calibrating an electronic device for characterizing a fluid with at least one sensor designed to interact with the fluid, by comparison with a reference electronic characterization device. It relates to a corresponding computer program, as well as a calibrated electronic device for characterizing a fluid by providing a signature obtained from an electrical measurement signal.

The invention applies more particularly to a calibration method comprising the following steps:
storing, in memory, a plurality of reference calibration signatures relating respectively to an interaction of a plurality of predetermined calibration fluids with at least one sensor of the reference electronic device;
storing, in memory, a parametric model, with at least one adjustable parameter, of the drift of the electronic device to be calibrated with respect to the reference electronic device;
for each of the plurality of predetermined calibration fluids, obtaining a calibration signature by interaction with said at least one sensor of the electronic device to be calibrated;
determining at least one value of said at least one adjustable parameter by comparison of the calibration signatures obtained with reference calibration signatures;

Thus, by applying said at least one determined value of adjustable parameter in the parametric drift model, it is possible to correct any signature obtained by interaction of any fluid with said at least one sensor of the electronic device as calibrated in this way.

Such a calibration procedure is taught, for example, in the article by Zhang et al., titled “On-line sensor calibration transfer among electronic nose instruments for monitoring volatile organic chemicals in indoor air quality,” published in Sensors and Actuators B: Chemicals, volume 160, issue 1, pages 899-909, Dec. 15, 2011. In this article, a global affine transformation drift model is used, based on the assumption of linear homogeneity between electronic devices for olfactory characterization of fluids. The hypothesis proves valid, but the reality of the drifts unfortunately does not quite follow the model.

To refine the parametric drift model, it can be made more complex. But it also makes calibration more complicated

It may therefore be desirable to provide a calibration method that avoids at least some of the above-mentioned problems and constraints.

A method is therefore proposed for calibrating an electronic device for characterizing a fluid with at least one sensor designed to interact with the fluid, by comparison with a reference electronic characterization device, comprising the following steps:
storing, in memory, a plurality of reference calibration signatures relating respectively to an interaction of a plurality of predetermined calibration fluids with at least one sensor of the reference electronic device;
storing, in memory, a parametric model, with at least one adjustable parameter, of the drift of the electronic device to be calibrated with respect to the reference electronic device;
for each of the plurality of predetermined calibration fluids, obtaining a calibration signature by interaction with said at least one sensor of the electronic device to be calibrated;
determining at least one value of said at least one adjustable parameter by comparison of the calibration signatures obtained with reference calibration signatures;
wherein the predetermined calibration fluids are divided into a number of different calibration groups and the determination of said at least one value of said at least one adjustable parameter is carried out specifically for each calibration group.

Thus, by cleverly providing several different calibration groups for which the respective parameter values of the parametric drift model are specific, and therefore a priori different from one calibration group to another, the calibration method is made finer, closer to the reality of possible drifts, without the need to complicate the parametric drift model to be used. Substantial improvements have been measured experimentally. It should further be noted that this calibration method is applicable both to the drift over time of a single electronic fluid characterization device, that is, the drift in repeatability of this device, and to the manufacturing drift of several electronic fluid characterization devices, that is, the drift in reproducibility of these devices.

Optionally, a calibration method according to the invention can further comprise a step of applying said at least one adjustable parameter value in the parametric drift model to correct any signature obtained by interaction of any fluid with said at least one sensor of the electronic device to be calibrated, this step comprising the selection of a calibration group to be associated with any fluid based on its signature, then the correction of its signature using said at least one adjustable parameter value of the selected group. It is indeed clever to associate, with the above-mentioned principle of calibration by differentiated groups, a step of prior selection of a calibration group for any fluid to be characterized based on its signature, even before correcting this signature by calibration, taking into account the drift model with the specificities of the selected group.

Also optionally, the selection of a calibration group to be associated with any fluid involves automatic classification of its signature in one of the calibration groups by comparison of this signature with the calibration signature(s) obtained from the calibration fluid(s) in each calibration group.

Also optionally, the correction of any fluid signature comprises an inversion of the parametric drift model in which said at least one adjustable parameter value of the selected calibration group is applied.

Also optionally, obtaining a calibration signature for each of the plurality of predetermined calibration fluids comprises obtaining a first calibration signature with N component(s), N≥1, then transforming this first calibration signature by normalization and/or component reduction.

Also optionally, the parametric drift model is an adjustable two-parameter affine model, where YDEV_{r,i}=αᵣ·YREF_{r,i}+tᵣ, where YDEV_{r,i} is the obtained calibration signature of the predetermined calibration fluid of index i of the calibration group of index r, YREF_{r,i} is the reference calibration signature of the predetermined calibration fluid of index i of the calibration group of index r, αᵣ is the value of a first adjustable multiplicative parameter of the affine model for the calibration group of index r and tᵣ is the value of a second adjustable additive parameter of the affine model for the calibration group of index r.

Also optionally, the correction of the signature of any fluid comprises the calculation YCOR=(YDEV−t_{\hat{r}})/α_{\hat{r}}, where YDEV is the signature obtained by interaction of any fluid with said at least one sensor of the electronic device to be calibrated, YCOR is the signature of any fluid after correction and is the index of the selected calibration group.

Also optionally, one of the first adjustable multiplicative parameter and the second adjustable additive parameter is independent of the calibration groups.

Also proposed is a computer program downloadable from a communications network and/or recorded on a computer-readable medium and/or executable by a processor capable of exchanging data with a memory in which are stored:
a plurality of reference calibration signatures relating respectively to an interaction of a plurality of predetermined calibration fluids with at least one sensor of a reference electronic characterization device;
a parametric model, with at least one adjustable parameter, of the drift of an electronic characterization device to be calibrated with respect to the reference electronic device;
the processor comprising instructions for performing the following steps:
for each of the plurality of predetermined calibration fluids, obtaining a calibration signature by interaction with at least one sensor of the electronic device to be calibrated;
determining at least one value of said at least one adjustable parameter by comparison of the calibration signatures obtained with reference calibration signatures;
wherein, the predetermined calibration fluids being divided into a number of different calibration groups, the instructions are more specifically designed so that the determination of said at least one value of said at least one adjustable parameter is carried out specifically for each calibration group.

Also proposed is a calibrated electronic device for characterizing a fluid by providing a signature obtained from an electrical measurement signal, comprising:
at least one sensor designed to interact with the fluid;
a transducer designed to provide, in interaction with said at least one sensor, the electrical measurement signal;
a storage memory:
of a parametric model, with at least one adjustable parameter, of the drift of the calibrated electronic device in relation to a reference electronic characterization device,
of at least one value of said at least one adjustable parameter, calculated by prior calibration of the calibrated electronic device by comparison with the reference electronic device based on a plurality of predetermined calibration fluids; and
a processor for correcting the signature by applying said at least one adjustable parameter value;
wherein:
the predetermined calibration fluids are divided into several different calibration groups and the memory stores at least one specific value of said at least one adjustable parameter for each of the calibration groups; and
the processor is more specifically programmed to select a calibration group to be associated with the fluid to be characterized based on its signature, then to correct its signature using said at least one specific adjustable parameter value of the selected group.

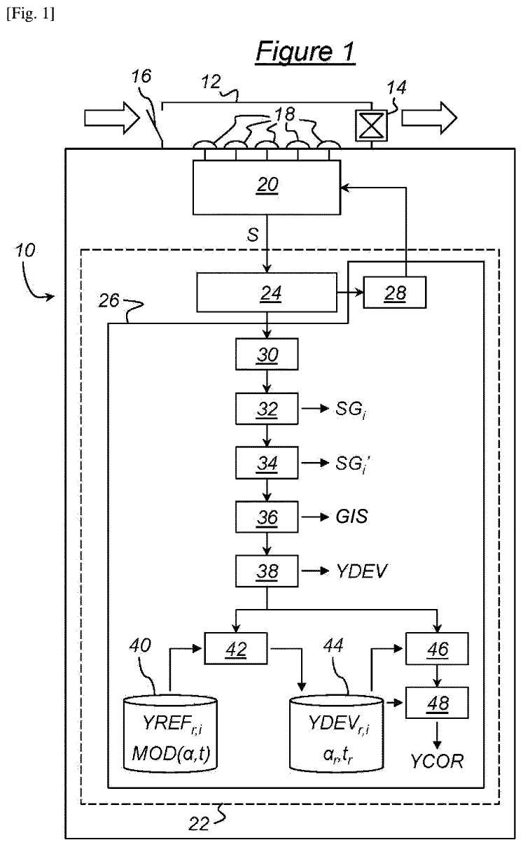

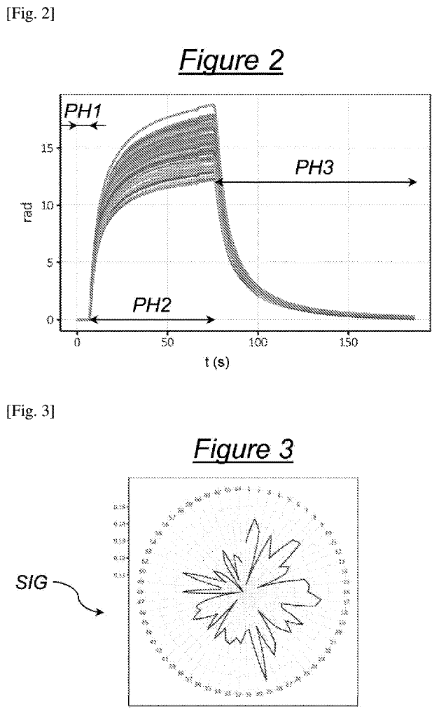

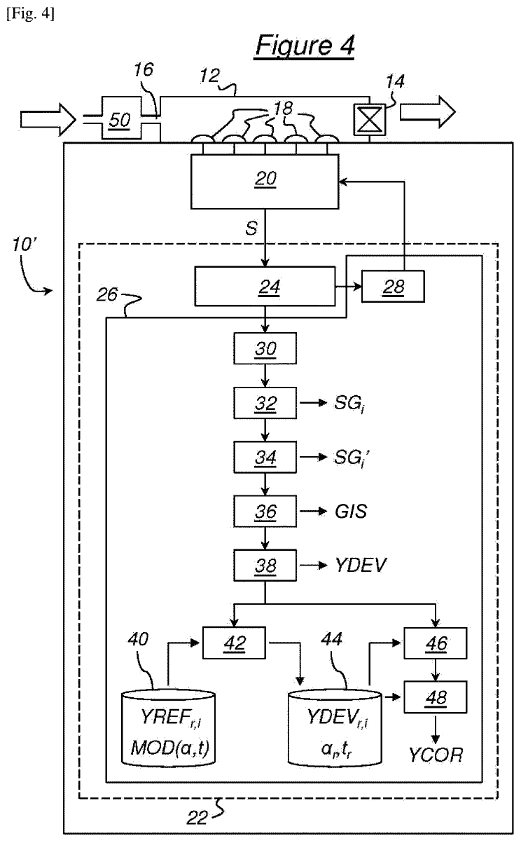

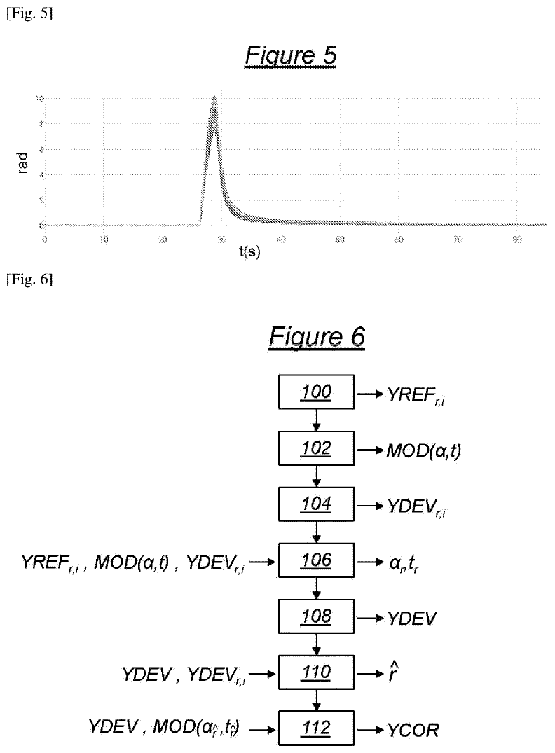

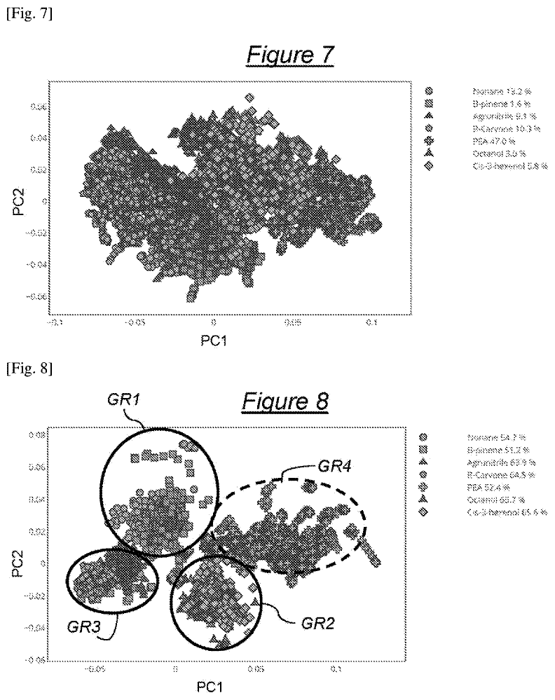

The invention will be better understood with the aid of the following description, given solely by way of example and done with reference to the appended drawings, wherein:

[FIG. 1] schematically depicts the general structure of a calibrated electronic device for characterizing a fluid, according to a first embodiment of the invention,

[FIG. 2] shows an example of the superposition of response signal time diagrams, or sensorgrams, that can be obtained by the device shown in [FIG. 1],

[FIG. 3] shows, in the form of a pie chart, an example of a normalized olfactory signature that can be calculated by the device of [FIG. 1] from sensorgrams such as those of [FIG. 2],

[FIG. 4] schematically depicts the general structure of a calibrated electronic device for characterizing a fluid, according to a second embodiment of the invention,

[FIG. 5] shows an example of the superposition of response signal time diagrams, or sensorgrams, that can be obtained by the device shown in [FIG. 4],

[FIG. 6] shows the successive stages of a calibration method to obtain the calibrated electronic device of [FIG. 1] or 4, according to one embodiment of the invention,

[FIG. 7] shows a diagram of experimental results that can be obtained without calibration according to the general principles of the present invention, and

[FIG. 8] shows a diagram of experimental results that can be obtained by applying calibration according to the general principles of the present invention.

The electronic device 10 for olfactory characterization of a fluid by providing a SIG signature obtained from an electrical measurement signal S, as depicted schematically in [FIG. 1], is a non-limiting example of an electronic fluid characterization device according to a first embodiment of the present invention for a non-limiting application of odor identification by multivalued olfactory measurement. It comprises a measuring chamber 12 designed to receive a fluid, for example a gas such as ambient air. To this end, it comprises a suction device 14 designed to draw air from inside the measuring chamber 12 and expel it to the outside. It further comprises an air inlet 16 that can be selectively closed to keep ambient air in the measuring chamber 12, or opened to allow ambient air to be discharged from the measuring chamber 12 and renewed by activating the suction device 14. It is thus equipped with the means to control incoming and outgoing flows.

In its measuring chamber 12, The electronic olfactory characterization device 10 comprises several sensors, in particular olfactory sensors 18, distributed respectively over as many reactive sites, for example around sixty, designed to interact with compounds likely to be present in the measuring chamber 12 when the device 10 is placed proximate to a fluid to be analyzed emitting these compounds, in particular when the air inlet 16 is proximate to the fluid in question. The compounds emitted are generally volatile organic compounds, but the present invention is not limited to such compounds.

Each olfactory sensor 18 is itself, for example, a biosensor designed to interact with compounds from a particular family of volatile organic compounds. In practice, each olfactory sensor 18 may comprise a molecule, such as a peptide immobilized on a substrate or a polymer covering a surface, complementary to the compounds of the family associated with this olfactory sensor 18.

Alternatively, the electronic olfactory characterization device 10 could be adapted to be brought into contact with any fluid, liquid or gaseous, other than ambient air. In a particularly simple version, it could also not comprise the suction device 14 and the air inlet 16, or even the measuring chamber 12. In this simple version, the olfactory sensors 18 can be brought into direct contact with the fluid to be analyzed, without flow control.

The olfactory sensors 18 are associated with and interact with at least one transducer 20. This transducer 20 is arranged and configured to measure any change in physical property caused by interaction of the olfactory sensors 18 with the fluid to be analyzed. It provides the electrical measurement signal S, for example in the form of a sequence of electrical measurement signals, and characterizes this fluid, since this sequence is representative of the volatile organic compounds with which the olfactory sensors 18 may interact in the measuring chamber 12.

More specifically, the transducer 20 can be a surface plasmonic resonance (SPR) imaging system configured to measure any change in refractive index due to an interaction of the fluid under study with at least one of the olfactory sensors owing to a plasmonic effect. Such a transducer comprises: a metal layer, a first face of which with reactive sites serves as a support for the olfactory sensors 18; an optical prism arranged against a second face, opposite the first, of the metal layer; a device for illuminating this second face of the metal layer with collimated and polarized light via a light input face of the optical prism; and a camera arranged at the light output of the optical prism for providing the sequence S of measurement signals in the form of a sequence of grayscale images of the reactive sites where the olfactory sensors 18 are arranged. For example, the reactive sites on the first side of the metal layer are organized in a positioning matrix grid.

Alternatively, the transducer 20 can be a Mach-Zehnder interferometer optical index variation amplification system, e.g. Mach-Zehnder Interferometer (MZI) matrix technology, or more specifically MZI Multi Mode Interference (MZI/MMI) technology. Such a system is configured to measure any change in refractive index due to interaction of the fluid under study with at least one of the olfactory sensors owing to a detectable phase shift between a reference arm of the interferometer and a sensing arm on which any such reactive site is arranged. The resulting transducer provides the sequence S of measurement signals in the form of a sequence of images of phase shifts, expressed in Radian, of the olfactory sensors 18.

In another variant, the transducer 20 could be a NEMS (Nano Electro-Mechanical System) or MEMS (Micro Electro-Mechanical System) amplification system. Such a system is configured to measure any change in the resonant frequency of a vibrating membrane whereupon any one of the olfactory sensors is arranged. The reactive sites on which the olfactory sensors 18 are placed are, for example, arranged in a matrix of NEMS or MEMS vibrating membranes for the provision of the measurement signal sequence S, which takes the form of a sequence of resonant frequency shift signals of the olfactory sensors 18.

Other alternatives are conceivable by implementing any other equivalent physical transduction device (that is, optical, mechanical, etc.), with a simple adaptation of the electronic olfactory characterization device 10 which will not be disclosed because it is within the reach of the skilled artisan.

Whatever the choice of transducer 20, the general idea remains to functionalize reactive sites using olfactory sensors 18 (that is, biosensors, polymers, carbon nanotubes, etc.) in such a way that they adsorb and desorb volatile organic compounds in a differentiated manner, to generate a differentiated molecular interaction response from the olfactory sensors, and to amplify the response in the form of a sequence S of electrical measurement signals using a physical transduction device.

The electronic olfactory characterization device 10 further comprises several functional modules which will be disclosed below. In the disclosed example, these modules are software modules. Thus, the device 10 comprises a computer-like element 22 comprising a processing unit 24 and an associated memory area 26 wherein several computer programs or several functions of the same computer program are stored. These computer programs comprise instructions designed to be executed by the processing unit 24 so as to perform the functions of the software modules. They are presented as distinct, but this distinction is purely functional. They could just as easily be grouped in any combination into one or more software packages. Their functions could also be at least partly micro-programmed or micro-wired into dedicated integrated circuits, such as digital circuits. Alternatively, the computer 22 could be replaced by an electronic device made up solely of digital circuits (without a computer program) to perform the same functions.

The electronic olfactory characterization device 10 thus firstly comprises a software module 28, to be executed by the processing unit 24, for controlling the suction device 14 (if provided in the device 10), the air inlet 16 (if also provided in the device 10) and the transducer 20.

Optionally, but advantageously, it further comprises a software module 30, to be executed by the processing unit 24, for selecting, from among the olfactory sensors 18 of the electronic olfactory characterization device 10, a subset of sensors sensitive to volatile components characteristic of a desired olfactory fingerprint. These characteristic volatile components can vary from one application or studied fluid to another, so that the selection of olfactory sensors made by software module 30 can also vary and be parameterized. The selected subset comprises, for example, M≥1 olfactory sensor(s), advantageously several olfactory sensors (M≥2).

The electronic olfactory characterization device 10 further comprises a software module 32, to be executed by the processing unit 24, for extracting M sensorgrams SGᵢ, i∈{1 . . . , M} respectively representative of the interactions of the M selected olfactory sensors with the relevant volatile organic compounds from the values specific to these M olfactory sensors in the sequence S of measurement signals supplied by the transducer 20. These sensorgrams SGᵢ, i∈[1, . . . , M] are, for example, reflectance signals expressed as a percentage, based on a ratio of luminance values obtained with transversely polarized light to luminance values obtained with the same light polarized at 90 degrees, for each of the M selected olfactory sensors when the transducer 20 is an SPR-type imaging system. They take the form of phase-shift signals, and are expressed in Radian when the transducer is an MZI or MZI/MMI amplification system. They take the form of frequency shift signals, and are expressed in Hertz when the transducer is a NEMS or MEMS amplification system.

FIG. 2 thus shows the superimposed time diagrams of some sixty sensorgrams SGᵢ, i∈{1, . . . . M} obtained using an MZI or MZI/MMI amplification system over a period of approximately 190 seconds according to a well-controlled measurement protocol involving control of the suction device 14 and air inlet 16, wherein:
the olfactory sensors 18 are first exposed to a carrier fluid reference fluidic environment without the presence of the target compounds of a fluid to be analyzed during a first reference state identifiable by a first portion PH1 of the sensorgrams,
they are then exposed to the fluid to be analyzed during a second analytical state of adsorption triggered by controlled injection of this fluid into the measuring chamber 12, this second state being identifiable by a second portion PH2 of the sensorgrams, and
they are finally re-exposed to the reference fluidic environment during a third final desorption state by controlled evacuation of the fluid to be analyzed from the measuring chamber 12, this third state being identifiable by a third portion PH3 of the sensorgrams.

Returning to FIG. 1, the electronic olfactory characterization device 10 further comprises an optional software module 34, to be executed by the processing unit 24, to perform any pre-processing on the M sensorgrams SGᵢ, i∈{1, . . . , M} provided by the software module 32.

This pre-processing comprises, for example, low-pass filtering in the form of a digital filter with finite or infinite impulse response. This involves filtering out the high-frequency measurement noise in the raw signals provided by the software module 32. A first-order Butterworth filter with a finite impulse response and a normalized cutoff frequency of 0.45 (that is, the ratio between the cutoff frequency and the sampling frequency equal to 0.45) is suitable.

This pre-processing for example further comprises a norm calculation within the meaning of patent document WO 2020/141281 A1 on the M filtered or unfiltered sensorgrams SGᵢ, i∈{1, . . . , M} to obtain M filtered and/or normalized sensorgrams SGᵢ, i∈{1, . . . , M}.

The electronic olfactory characterization device 10 further comprises a software module 36, to be executed by the processing unit 24, to obtain in a well-known and non-detailed way a characterization or GIS signature of the composition of the fluid to be analyzed from the M sensorgrams SGᵢ, i∈{1, . . . , M} or SGᵢ, i∈{1, . . . , M}. This characterization or GIS signature can take the form of a standardized olfactory signature as shown in [FIG. 3] in the form of a pie chart. Note that this module can proceed in two stages: first, obtaining a first intermediate signature, then transforming this first intermediate signature by normalization.

The electronic olfactory characterization device 10 further comprises an optional software module 38, to be executed by the processing unit 24, for transforming the GIS signature, standardized or not, into another YDEV signature simplified by component reduction. Linear Discriminant Analysis (LDA), Principal Component Analysis (PCA), Independent Component Analysis (ICA), auto-encoder, etc., are all suitable. For a sixty-four-component GIS signature such as that shown in [FIG. 3], a simplified two- or three-component YDEV signature can be obtained.

The electronic olfactory characterization device 10 further comprises a portion 40 of the memory area 26 for storing a parametric model MOD, with at least one adjustable parameter, of drift relative to a reference electronic characterization device. This parametric drift model is, for example, an affine model MOD(α,t) with two adjustable parameters α and t, where α is a first adjustable multiplicative parameter of the affine model and t is a second adjustable additive parameter of the affine model. It should be noted that the reference characterization electronic device may be an electronic device specifically identified as a standard, a set of electronic devices specifically identified as standards, or a virtual electronic device resulting from such a set, for example by means of average, median or any other relevant aggregation calculation.

The portion 40 of the memory area 26 further stores a plurality of reference calibration signatures relating respectively to an interaction of a plurality of predetermined calibration fluids with at least one sensor of the reference electronic device. For the sake of consistency, the reference device comprises the same olfactory sensors as the electronic olfactory characterization device 10. In accordance with the general principles of the present invention, the predetermined calibration fluids are divided into several different calibration groups. This means that if there are any number G≥2 of different calibration groups, then there is a number greater than or equal to G of calibration fluids distributed in these G calibration groups at a rate of at least one calibration fluid per group. Distribution can be random, by signature similarity according to a similarity criterion, or any other distribution rule within the reach of the skilled artisan. For example, YREF_{r,i} is the simplified reference calibration signature of the predetermined calibration fluid of index i of the calibration group of index r. Each signature YREF_{r,i} may, for example, be the result of an aggregation calculation of measurement values taken on several electronic fluid characterization devices, such as an average, a median, a maximum, a minimum or the like. The reference electronic device then represents a theoretical, that is, virtual, aggregation of these electronic fluid characterization devices.

The electronic olfactory characterization device 10 further comprises a software module 42, to be executed by the processing unit 24, to perform calibration by comparison with the reference electronic characterization device. This calibration software module 42 receives as input calibration signatures obtained by interaction of predetermined calibration fluids with the olfactory sensors 18 of the electronic olfactory characterization device 10, and stores them in a portion 44 of the memory area 26. These calibration signatures, specific to the electronic olfactory characterization device 10, are more specifically obtained by running software modules 28 to 38 on each of the predetermined calibration fluids. For example, YDEV_{r,i} is the obtained simplified calibration signature of the predetermined calibration fluid of index i of the calibration group of index r.

In accordance with the general principles of the present invention, calibration consists in determining a value for each of the adjustable parameters, in this case α and t for the aforementioned affine model, specifically for each calibration group. More specifically, the aim here is to determine each pair of specific values (αᵣ, tᵣ) for each calibration group of index r, such that it can be considered that for each YDEV_{r,i}=αᵣ·YREF_{r,i}+tᵣ for each predetermined calibration fluid of index i of each calibration group of index r. This determination is carried out by the software module 42, using a known optimization software method such as squared error minimization or maximum likelihood estimation. It then stores the result, that is, the pairs of specific values (αᵣ, tᵣ) of the different calibration groups, in the portion 44 of the memory area 26. The electronic olfactory characterization device 10 can then be considered calibrated.

Note that the parameters αᵣ and tᵣ can take scalar values, but also vector values with as many components as the signatures YDEV_{r,i}et YREF_{r,i}.

It should also be noted that, in accordance with the general principles of the present invention, one of the first adjustable multiplicative parameter α and the second additive parameter t is independent of the calibration groups. In this way, the adjustable multiplicative parameter α can take on different values αᵣ from one calibration group to the next, while the adjustable additive parameter t remains constant at t₀, and t₀ can even be zero. Similarly, the adjustable additive parameter t can take on different values tᵣ from one calibration group to the next, while the adjustable multiplicative parameter α remains constant at α₀, α₀ possibly even being neutral at 1.

The electronic olfactory characterization device 10 further comprises two software modules 46 and 48, to be executed by the processing unit 24, to perform a correction on the measured signature of any fluid to be characterized. More specifically, the software module 46 selects a calibration group to be associated with the fluid to be characterized based on its signature, while the software module 48 corrects this signature using the specific values of the adjustable parameters of the selected group.

Specifically, the selection software module 46 receives an input signature, for example a simplified signature YDEV, obtained by interaction of the fluid to be analyzed with the olfactory sensors 18 of the electronic olfactory characterization device 10. More specifically, this signature is obtained by running software modules 28 to 38 on the fluid to be characterized. The selection software module 46 then compares this simplified signature YDEV with the calibration signatures YDEV_{r,i} and selects the calibration group, with index r, that seems most representative of this signature according to a predetermined criterion. This can be achieved by a number of well-known, more or less simple methods. Advantageously, the selection is made by automatically classifying the simplified signature YDEV in one of the calibration groups by comparing this signature with the calibration signature(s) obtained from the calibration fluid(s) in each calibration group. In particular, a k-nearest neighbors method (k≥1) is ideally suited to the situation.

The correction software module 48 receives the index f of the group selected as input and applies the corresponding parameter values % and 4 to an inversion of the parametric drift model. In concrete terms, in the example of the affine model mentioned above, this means applying the following correction to the signature YDEV:

$$YCOR = \frac{( YDEV \text{?} )}{\text{?}} .$$

$$\text{?} \text{indicates text missing or illegible when filed}$$

YCOR is therefore the simplified signature of the fluid to be characterized after correction.

According to a second embodiment of the present invention, the electronic device 10′ for olfactory characterization of a fluid shown schematically in [FIG. 4] differs from that of [FIG. 1] in that the air inlet 16 is connected to a thermodesorption concentrator 50 by means of which it receives the fluid to be analyzed. All other components of the electronic device 10′ remain identical to those of the electronic device 10 so that they retain the same references. The thermodesorption concentrator 50 is a well-known device that will not be detailed here. It works by accumulating compounds in a fluid by adsorption onto a resin. The resin is then heated to a set temperature, e.g. 200° C., to trigger desorption of these compounds from the resin. Once the set temperature has been reached, the compounds are injected into the electronic device 10′.

This results in the rapid injection of a concentration peak rather than the continuous injection of compounds in the context of a measurement as disclosed in the previous embodiment. This concentration peak then generates distinct temporal signals in the form of peaks, rather than the temporally extended sensorgrams of [FIG. 2]. However, by a legitimate misuse of language, these distinct temporal signals can also be called sensorgrams, since they are still a response of the transducer 20 to the introduction of a fluid to be analyzed into the measuring chamber 12, even if this introduction takes place in a different way.

FIG. 5 thus shows the superimposed time diagrams of some sixty sensorgrams SGᵢ, i∈{1 . . . , M} obtained using an MZI or MZI/MMI amplification system over a period of around 80 seconds, according to a well-controlled measurement protocol using the thermodesorption concentrator 50.

The successive steps of a calibration method for the electronic olfactory characterization device 10 or 10′ will now be detailed with reference to [FIG. 6].

In a step 100, the reference signatures YREF_{r,i} of the predetermined calibration products are stored in the memory 40 of the electronic olfactory characterization device 10 or 10′. They are acquired by executing software modules, equivalent to software modules 28 to 38, of an electronic reference device, virtual or otherwise, similar to the electronic olfactory characterization device 10 or 10′.

In a step 102 performed before, during or after step 100, the parametric drift model MOD(α,t) of the olfactory characterization electronic device 10 or 10′ to be calibrated with respect to the reference electronic device is also stored in memory 40.

In a step 104 carried out by running software modules 28 to 38, the calibration signatures YDEV_{r,i} of predetermined calibration products are obtained by interaction with the sensors 18 of the electronic olfactory characterization device 10 or 10′.

In a step 106 performed by running the software module 42, the calibration signatures YDEV_{r,i} are stored in memory 44. In the same step, the specific values (αᵣ, tᵣ) of the adjustable parameters of the parametric model MOD(α,t) are determined and stored in memory 44 for each of the different calibration groups.

At the end of this step, the electronic olfactory characterization device 10 or 10′ can be considered calibrated.

In a subsequent step 108, carried out by running software modules 28 to 38, a fluid to be characterized is brought near the electronic olfactory characterization device 10 or 10′ for interaction with its olfactory sensors 18. The result is the simplified signature YDEV.

In a subsequent step 110, carried out by running software module 46, the calibration group of index r considered most representative of the simplified signature YDEV is selected.

Finally, in a last step 112, carried out by running the software module 48, this signature YDEV is corrected at

$$YCOR = \frac{( YDEV \text{?} )}{\text{?}}$$

$$\text{?} \text{indicates text missing or illegible when filed}$$

by inversion of the parametric drift model.

It should be noted that the aforementioned calibration method provides for a possible simplification of the signatures by dimension reduction prior to application of the calibration parameters, so that the latter are then adapted to the reduced dimension after simplification when they are vector-based. Alternatively, calibration can be carried out before dimension reduction, so that the calibration parameters are adapted to the initial dimension before simplification if they are vector-based.

The diagrams in FIGS. 7 and 8 show experimental results obtained on predetermined fluidic samples whose signatures are measured on a large number of different electronic characterization devices. Signatures are simplified by principal component analysis, and two first principal components PC1, PC2 are retained to characterize the samples. Seven sample types are available for measurement: nonane samples, β-pinene samples, agrunitrile samples, R-carvone samples, PEA (phenylethyl alcohol) samples, octanol samples and cis-3-hexenol samples.

FIG. 7 shows the distribution of various simplified signatures obtained, without any prior calibration, in the principal component planes PC1 and PC2. A Clustering Quality Score (CQS), as taught for example in patent document WO 2022/053690 A1, can be calculated for each of the sample types proposed for measurement and averaged over all the samples. Clearly, it is difficult to characterize the different types of sample in [FIG. 7]. Experimentally, nonane samples had a CQS score of 13.2%, β-pinene samples a CQS score of 1.6%, agrunitrile samples a CQS score of 9.1%, R-carvone samples a CQS score of 10.3%, PEA samples a CQS score of 47%, octanol samples a CQS score of 3.0% and cis-3-hexenol samples a CQS score of 5.8%, for an overall average CQS score of 12.9%. This is not very satisfactory.

When a calibration according to the present invention is carried out, a result like the one shown in the diagram in FIG. 8 can be obtained. Three calibration groups are defined, for example: a first group made up of nonane and β-pinene samples, a second group of octanol and cis-3-hexenol, and a third group of agrunitrile and R-carvone. PEA samples, for example, are not used for calibration. The parametric drift model chosen is an affine model wherein the values αᵣ of the multiplicative parameter α are scalar and the values tᵣ of the additive parameter t are vector-based. FIG. 8 shows the distribution of various simplified and corrected signatures obtained, after the aforementioned calibration, in the plane of principal components PC1 and PC2. More specifically, FIG. 8 shows a case of simplification of the signatures after calibration, so that the vector values tᵣ of the additive parameter t are of the dimension of the signatures before dimension reduction. Four groups of signatures stand out more clearly than in [FIG. 7]. A first group GR1 contains most of the nonane and β-pinene samples. A second group GR2 contains most of the octanol and cis-3-hexenol samples. A third group GR3 contains most of the agrunitrile and R-carvone samples. A fourth group GR4, shown in dotted lines because it contains compounds that were not part of a calibration group, contains most of the PEA samples. Logically, nonane samples experimentally show an improved CQS score of 54.7%, β-pinene samples an improved CQS score of 51.2%, agrunitrile samples an improved CQS score of 63.9%, R-carvone samples an improved CQS score of 64,5%, PEA an improved CQS score of 52.4%, octanol an improved CQS score of 65.7% and cis-3-hexenol an improved CQS score of 65.6%, for an overall significantly improved mean CQS score of 59.7%.

It is clear that a calibration method such as the one described above can effectively compensate for drifts in the performance of electronic fluid characterization devices, both over time and from one device to the next. This improves discrimination of the fluids to be characterized.

It should also be noted that the invention is not limited to the embodiments previously disclosed.

In particular, a single parametric drift model has been presented, but other models may be considered depending on the reality of possible drifts. It should simply be noted that the greater the number of parameters to be set, the greater the number of calibration groups or the number of calibration fluids per group.

More generally, it will be apparent to the skilled artisan that various modifications can be made to the embodiments disclosed herein before, in view of the teachings just disclosed. In the above detailed presentation of the invention, the terms used are not to be construed as limiting the invention to the embodiments set out in the present disclosure, but are to be interpreted as including all equivalents the anticipation of which is within the reach of the skilled artisan by applying his general knowledge to the implementation of the teaching just disclosed to him.

1. A method of calibrating an electronic device for characterizing a fluid with at least one sensor designed to interact with the fluid, by comparison with a reference electronic characterization device, comprising the following steps:
storing, in memory, a plurality of reference calibration signatures (YREF_{r,i}) relating respectively to an interaction of a plurality of predetermined calibration fluids with at least one sensor of the reference electronic device;
storing, in memory, a parametric model (MOD(α,t)), with at least one adjustable parameter (α,t), of the drift of the electronic device to be calibrated with respect to the reference electronic device;
for each of the plurality of predetermined calibration fluids, obtaining a calibration signature (YDEV_{r,i}) by interaction with said at least one sensor of the electronic device to be calibrated;
determining at least one value (αᵣ,tᵣ) of said at least one adjustable parameter (α,t) by comparison of the calibration signatures (YDEV_{r,i}) obtained with reference calibration signatures (YREF_{r,i});
wherein the predetermined calibration fluids are divided into a number of different calibration groups and the determination of said at least one value (αᵣ, tᵣ) of said at least one adjustable parameter (α,t) is carried out specifically for each calibration group.

2. The calibration method according to claim 1, further comprising a step of applying said at least one adjustable parameter value (αᵣ, tᵣ) in the parametric drift model (MOD(α,t)) to correct any signature (YDEV) obtained by interaction of any fluid with said at least one sensor of the electronic device to be calibrated, this step comprising the selection of a calibration group (\hat{r}) to be associated with any fluid based on its signature (YDEV), then the correction of its signature using said at least one adjustable parameter value of the selected group.

3. The calibration method according to claim 2, wherein the selection of a calibration group to be associated with any fluid involves automatic classification of its signature (YDEV) in one of the calibration groups by comparison of this signature with the calibration signature(s) obtained from the calibration fluid(s) in each calibration group.

4. The calibration method according to claim 2, wherein the correction of any fluid signature (YDEV) comprises an inversion of the parametric drift model (MOD(α,t)) in which said at least one adjustable parameter value of the selected calibration group is applied.

5. The calibration method according to claim 1, wherein obtaining a calibration signature (YDEV_{r,i}) for each of the plurality of predetermined calibration fluids comprises obtaining a first calibration signature with N component(s), N≥1, then transforming this first calibration signature by normalization and/or component reduction.

6. The calibration method according to claim 1, wherein the parametric drift model (MOD(α,t)) is an adjustable two-parameter affine model, YDEV_{r,i}=αᵣ·YREF_{r,i}+tᵣ, where YDEV_{r,i} is the obtained calibration signature of the predetermined calibration fluid of index i of the calibration group of index r, YREF_{r,i} is the reference calibration signature of the predetermined calibration fluid of index i of the calibration group of index r, αᵣ is the value of a first adjustable multiplicative parameter of the affine model for the calibration group of index r and tᵣ is the value of a second adjustable additive parameter of the affine model for the calibration group of index r.

7. The calibration method according to claim 2, wherein the correction of the signature (YDEV) of any fluid comprises the calculation YCOR=(YDEV−t_{\hat{r}})/α_{\hat{r}}, where YDEV is the signature obtained by interaction of any fluid with said at least one sensor of the electronic device to be calibrated, YCOR is the signature of any fluid after correction and f is the index of the selected calibration group.

8. The calibration method according to claim 6, wherein one of the first adjustable multiplicative parameter and the second adjustable additive parameter is independent of the calibration groups.

9. A computer program downloadable from a communications network and/or recorded on a computer-readable medium and/or executable by a processor capable of exchanging data with a memory in which are stored:
a plurality of reference calibration signatures (YREF_{r,i}) relating respectively to an interaction of a plurality of predetermined calibration fluids with at least one sensor of a reference electronic characterization device;
a parametric model (MOD(α,t)), with at least one adjustable parameter, of the drift of an electronic characterization device to be calibrated with respect to the reference electronic device;
the computer program comprising instructions for performing the following steps when executed on a computer:
for each of the plurality of predetermined calibration fluids, obtaining a calibration signature (YDEV_{r,i}) by interaction with at least one sensor of the electronic device to be calibrated;
determining at least one value (αᵣ, tᵣ) of said at least one adjustable parameter (α,t)) by comparison of the calibration signatures (YDEV_{r,i}) obtained with reference calibration signatures (YREF_{r,i});
wherein, the predetermined calibration fluids being divided into a number of different calibration groups, the instructions are more specifically designed so that the determination of said at least one value (αᵣ, tᵣ) of said at least one adjustable parameter (α,t) is carried out specifically for each calibration group.

10. A calibrated electronic device for characterizing a fluid by providing a signature (YDEV) obtained from an electrical measurement signal (S), comprising:
at least one sensor designed to interact with the fluid;
a transducer designed to provide, in interaction with said at least one sensor, the electrical measurement signal (S);
a storage memory:
a parametric model (MOD(α,t)), with at least one adjustable parameter (α,t), of the drift of the calibrated electronic device in relation to a reference electronic characterization device,
at least one value (αᵣ, tᵣ) of said at least one adjustable parameter (α,t), calculated by prior calibration of the calibrated electronic device by comparison with the reference electronic device based on a plurality of predetermined calibration fluids; and
a processor for correcting the signature (YDEV) by applying said at least one adjustable parameter value;
wherein:
the predetermined calibration fluids are divided into several different calibration groups and the memory stores at least one specific value (αᵣ, tᵣ) of said at least one adjustable parameter (α,t) for each of the calibration groups; and
the processor is more specifically programmed to select a calibration group to be associated with the fluid to be characterized based on its signature (YDEV), then to correct its signature using said at least one specific adjustable parameter value (αᵣ, tᵣ) of the selected group.
